(19) United States
(12) Patent Application Publication
(10) Pub. No.: US 2026/0203710 A1
(43) Pub. Date: Jul. 16, 2026

(54) MULTI-POINT CONNECTIVITY AMONG PROJECT STAKEHOLDERS

(71) Applicant: UES Professional Solutions, LLC, Orlando, FL (US)

(72) Inventors: David Witsken, Orlando, FL (US); Kathleen Kilmer, Orlando, FL (US); Sol Fontanes, Orlando, FL (US); Suresh Sanka, Lake Mary, FL (US); Alex Jaramillo, Burbank, CA (US)

(21) Appl. No.: 19/445,252

(22) Filed: Jan. 9, 2026

Related U.S. Application Data
(60) Provisional application No. 63/743,923, filed on Jan. 10, 2025.

(51) Int. Cl. G06Q 10/087; G16H 10/40; G16H 15/00

(52) U.S. Cl. CPC G06Q 10/0877; G16H 10/40; G16H 15/00

(57) ABSTRACT

Methods, systems, and apparatuses are disclosed for providing seamless integration among laboratory, office, field, and client operations. A platform features a portal for scheduling work orders and managing logistics of one or more tasks and tests associated with the work orders. Logging completion of tasks in the portal provides visibility into a project's timeline. User devices enable real-time field data capture and analysis. Embodiments of the invention incorporate logistics management, laboratory testing information integration, GIS capabilities, comprehensive project management tools, customizable form and report creation, and integration with client and third-party databases. Utilizing artificial intelligence, the system analyzes project data to reveal key performance indicators and predictive timeline analytics, presenting findings through an intuitive dashboard of the Portal. The invention enhances operational efficiency, data accuracy, and strategic decision-making by unifying disparate processes into a single, scalable platform.

### CROSS-REFERENCE TO RELATED APPLICATIONS AND INCORPORATION BY REFERENCE

This patent application is a non-provisional of and claims priority to U.S. Provisional Ser. No. 63/743,923 , filed in the United States Patent and Trademark Office (USPTO) on Jan. 10, 2025, entitled “MULTI-POINT CONNECTIVITY AMONG PROJECT STAKEHOLDERS”, the entire disclosure of which is incorporated herein by reference in its entirety.

### FIELD OF THE INVENTION

This disclosure relates to systems, methods, and apparatuses for providing multi-point connectivity among stakeholders on geotechnical and construction projects. For example, geotechnical engineering, environmental consulting, materials testing and inspection services. In particular, a cloud-based platform designed to provide seamless connectivity among laboratory, office, field, and client operations. This platform encompasses functionalities such as user portals, scheduling and logistics, field data capture and analysis, laboratory testing information management, Geographic Information System (GIS) integration, project management, form and report creation, integration with external databases, and the utilization of artificial intelligence for advanced data analytics and dashboard presentation.

### BACKGROUND

In various industries, efficient coordination between personnel in laboratories, offices, the “field”, and clients is crucial for successful project execution. Traditional systems often operate in silos, leading to fragmented data, communication gaps, and operational inefficiencies. Field data is frequently collected manually and entered into separate systems, causing delays and potential errors. Laboratories manage testing information independently, making it challenging to integrate test results promptly into project workflows. Moreover, the lack of advanced analytical tools hinders organizations from extracting meaningful insights from accumulated data, thereby negatively impacting decision-making processes.

Geotechnical and construction projects involving construction quality assurance routinely encompass on-site pouring of concrete and the contemporaneous preparation of standardized test specimens (most commonly cylinders) to verify material integrity and specification compliance through subsequent laboratory testing. During field operations, technicians observe the concrete placement, record project conditions, and cast one or more specimens from the poured concrete in molds bearing unique identifiers tied to the corresponding work order, thereby establishing an auditable chain of custody from field collection through laboratory testing and reporting. After initial field handling, specimens are left in a designated on-site location for pickup and transport to the laboratory. Upon receipt by a laboratory, laboratory personnel strip molds, transfer labels to the hardened specimens, and store the specimens until their scheduled “break” dates. At the scheduled age, lab technicians may perform standardized concrete strength tests (e.g., compressive testing of cylinders) and enter the data into reports for use by the project stakeholders.

Beyond concrete, geotechnical projects also require systematic characterization of soils used as engineered fill and subgrade, where soil density and moisture relationships govern bearing capacity, settlement performance, and constructability. Laboratory compaction testing produces a reference compaction curve that establishes a maximum dry density and corresponding optimal moisture content for a given soil under defined compactive effort.

Conventional practice coordinates work orders, pickups, tests, and reporting across dispatchers, field technicians, drivers or couriers, laboratory personnel, project managers, and client representatives through channels such as email, text messages, phone calls, and spreadsheets, rather than a unified system. Work orders are often issued and revised outside controlled workflows, resulting in inconsistent specimen identifiers; pickups depend on manual notifications and paper tickets, producing missed collections, improper curing durations, or misrouting of samples; chain-of-custody records are split between field forms and laboratory logs, creating reconciliation gaps; and test data frequently undergo multiple transcriptions from hand notes to bench sheets to formal reports, introducing potential transcription error. Review and approval of formal reports are prolonged because approvers lack real-time visibility into specimen status (cast, picked up, curing, queued, tested), equipment readiness, and schedule constraints, while clients receive piecemeal reports that lack traceable linkage between field observations and laboratory results. These coordination inefficiencies compound across projects, increasing re-test rates, delaying acceptance decisions, and elevating compliance risk under applicable standards and contract specifications.

Thus, there is a significant need for an integrated, cloud-based system that not only streamlines data capture and management but also orchestrates end-to-end workflows across various stakeholders; enforces specimen chain-of-custody, time-stamped geolocation, and photographic evidence; automates pickup scheduling, curing tracking, and laboratory test queues; and implements role-based review and distribution of final reports to clients. Such a system should provide real-time visibility into specimen and test status, support offline mobile data acquisition with subsequent synchronization to ensure timely, accurate, and compliant decision-making. Thus, materially improving the reliability and efficiency of geotechnical and construction quality assurance operations.

### BRIEF SUMMARY

This disclosure introduces methods and systems for comprehensive cloud-based platform designed to revolutionize the way laboratories, offices, field operations, and clients interact and collaborate. Accessible through both web-based interfaces and mobile applications, this unique platform provides seamless connectivity that unifies disparate operational areas into a cohesive ecosystem.

At its core, the invention addresses the inefficiencies and communication gaps prevalent in legacy systems by integrating a multitude of functionalities into a single, scalable solution. The platform offers a secure portal that facilitates direct engagement between clients and project personnel, enabling real-time access to project information, status, reports, and updates. It streamlines scheduling and logistics by providing tools for efficient planning and coordination of resources, thereby enhancing operational productivity.

The platform significantly improves field operations through its advanced data capture and analysis capabilities. Field personnel can collect data in real-time using mobile devices, even in offline environments, with immediate synchronization and analytical processing upon establishing internet connectivity.

Laboratory testing information is seamlessly integrated into the project workflow, ensuring timely access to results and enhancing data-driven decision-making. This technological integration provides real-time operational knowledge of projects by granting access to sample tracking, testing procedures, analysis results, reporting, and equipment utilization. Recognizing the critical role that material integrity (e.g. concrete) plays in the longevity and performance of structures, this system ensures the quality, safety, and compliance of construction projects.

A team of certified technicians and engineers may conduct on-site testing and inspection services. In embodiments, the platform enables laboratory information management for facilitating efficient sample tracking, status, analysis, and reporting. The system verifies that lab tests meet project specifications and adhere to industry standards and regulatory requirements. Services such as field density/soil compaction testing, concrete strength testing, and asphalt density testing may be tracked using the platform, providing timely and precise testing results. This integration helps clients identify potential issues early, reducing the risk of costly delays and ensuring project milestones are met.

In addition to field services, the platform enhances efficiency by automating data collection and analysis, and it streamlines reporting processes for laboratory facilities where materials (e.g. concrete, soil, asphalt, etc. . . . ) undergo analysis. This includes a broad spectrum of tests like, but not limited to, compressive strength tests for concrete, field density testing, and mix design evaluations for asphalt. Equipment utilization is optimized as the system monitors the status and maintenance schedules of laboratory instruments, thereby ensuring resources are used effectively.

Geographic Information System (GIS) integration allows for sophisticated spatial data visualization and analysis, supporting functions such as route optimization and mapping of specimen locations. The platform's project management tools offer comprehensive oversight of project timelines, document review & input, technical analysis, billing, work orders, tasks, and team collaboration, fostering an environment of transparency and accountability.

Furthermore, the platform facilitates the creation of customized forms and reports, standardizing documentation processes and ensuring compliance with organizational standards. Its ability to integrate with client and third-party databases ensures seamless data exchange and maintains data consistency across systems.

A feature of the invention is its utilization of artificial intelligence to analyze project data. AI algorithms provide insights into key performance indicators and predictive timeline analytics, enabling organizations to anticipate challenges and optimize operations proactively. The analyzed data is presented through an intuitive dashboard, offering customizable visualizations that support informed strategic decisions.

Embodiments of the invention transform operational workflows by bridging the gaps between various functional areas, enhancing data accuracy, and promoting efficient communication among all stakeholders involved in a project.

### BRIEF DESCRIPTION OF THE DRAWINGS

The accompanying drawings, which are incorporated into and form a part of the specification, illustrate one or more embodiments of the present invention and, together with the description, serve to explain the principles of the invention. The drawings are only for the purpose of illustrating exemplary embodiments of the invention and are not to be construed as limiting the invention. In the drawings:

FIG. 1 illustrates a flow diagram of an embodiment showing work order creation, acceptance, performance, report generation, and report delivery.

FIG. 2 illustrates a flow diagram of an embodiment covering work orders that involve on-site testing and specimen pickup/check-in processes through lab receipt and notifications.

FIG. 3 illustrates a flow diagram continuing from FIG. 2.

FIG. 4 illustrates a flow diagram of an embodiment addressing field-density workflows, including creation of a proctor report and subsequent field testing steps..

FIG. 5 illustrates a page of a web-based user interface (e.g., a dashboard/home page view).

FIG. 6 illustrates a page of a web-based user interface showing work-order listings and controls to create new work orders.

FIG. 7 illustrates a page of a web-based user interface for entering work-order details.

FIGS. 8A and 8B illustrate embodiments of a mobile application user interface showing a login screen and a modules landing page..

FIGS. 9A and 9B illustrate embodiments of a mobile application user interface showing a settings panel and a new work-order assignment notification.

FIGS. 10A and 10B illustrate embodiments of a mobile application user interface showing work-order list views and date-range selection.

FIGS. 11A and 11B illustrate embodiments of a mobile application user interface showing calendar filtering and additional list views.

FIGS. 12A and 12B illustrate embodiments of a mobile application user interface showing a work-detail view with editable fields.

FIGS. 13A and 13B illustrate embodiments of a mobile application user interface showing location/map features and a sample pick-up form.

FIG. 14 illustrates a schematic architecture of the system.

FIG. 15 illustrate an assignment view for accepting work orders.

FIG. 16 illustrates an exemplary concrete sampling and testing report form.

FIGS. 17-18 illustrate embodiments of platform user interfaces.

FIG. 19 illustrates an automatically created hierarchical folder structure for proposals within a project-focused configuration.

FIG. 20 illustrates an automatically created hierarchical folder structure for projects following award (“Won” proposals) in a project-focused configuration.

FIG. 21 illustrates a client-focused hierarchical folder structure for proposals and projects.

FIG. 22 illustrates an automatically created hierarchical folder structure for projects following award (“Won” proposals) in a client-focused configuration.

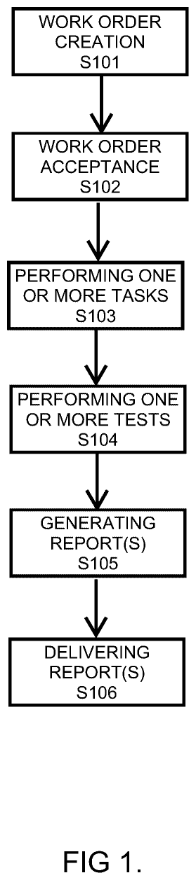

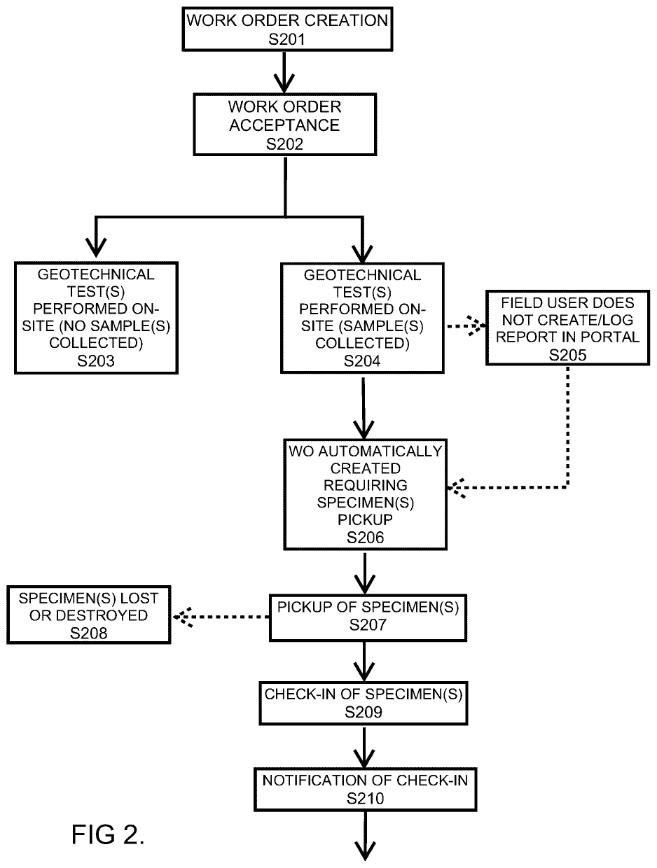

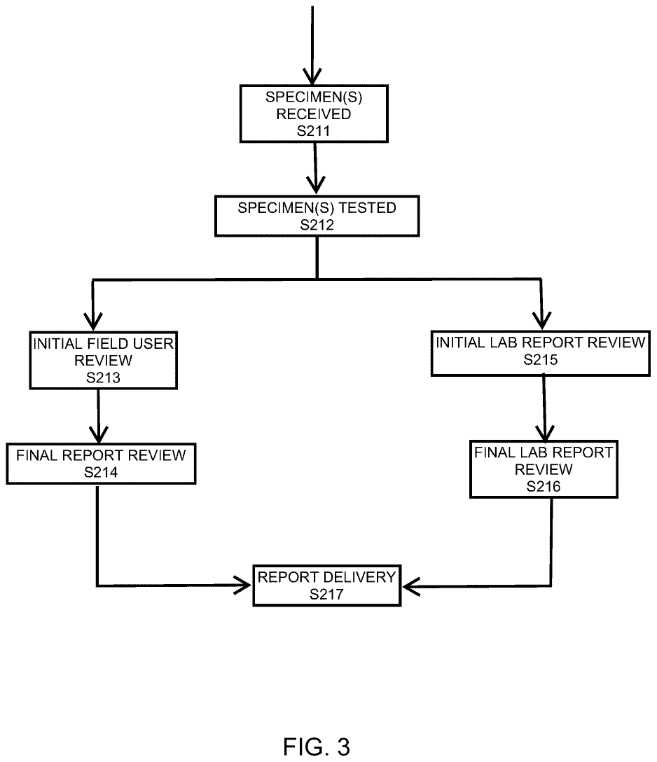

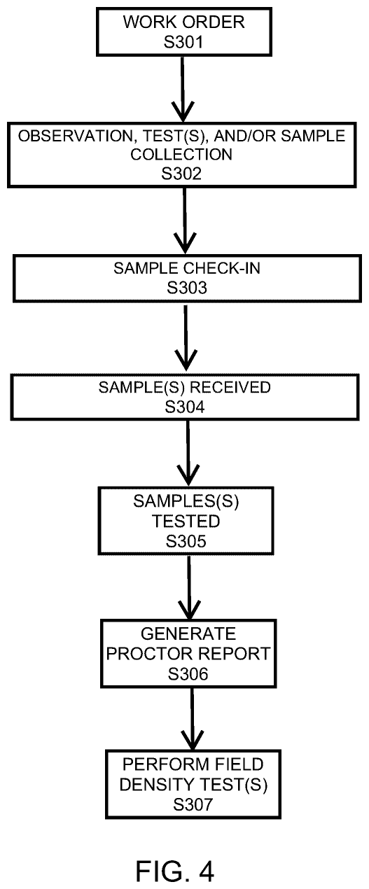

### DETAILED DESCRIPTION

In embodiments, the invention comprises a cloud-based platform engineered to unify and enhance the operational efficiency of laboratories, offices, field operations, and client interactions. The platform is designed to cater to the specific needs of different project stakeholders while maintaining a user-friendly interface.

As used herein, the term “Office User” means a user of the platform who typically sits in an office and handles administrative work associated with a project.

As used herein, the term “Field User” means a user of the platform who are on-site at a project, colloquially referred to as the “Field”. Field users include, but are not limited to, Field Technicians, Project Managers, and Inspectors.

As used herein, the term “Lab User” means a user of the platform who performs one or more laboratory duties in relation to a project. Lab Users include, but are not limited to, Lab Technicians and Lab Managers.

As used herein, the term “Client” or “Client User” means one or more end customers who contract with engineering companies.

As used herein, the terms “Project Field Representative”, “PFR”, broadly means field representatives, for example but not limited to, Site Superintendents, Governing body Inspectors, and/or Client Representatives.

As used herein, the terms “specimen” and “sample” may be used interchangeably and, unless the context clearly indicates otherwise, refer to any collected material unit or test article obtained, prepared, handled, transported, stored, tested, analyzed, or reported within the workflows described, including without limitation concrete cylinders or beams, soil materials, and other project-related materials subject to testing, and reporting in the platform; references to the singular include the plural and vice versa, and capitalization does not denote a substantive distinction in meaning

As used herein, “software” and “code” include within their meaning one or more lines of computer readable and executable instructions that are stored in, and able to be read from, computer storage media that is in communication with a processor. The processor, or controller, reads the software from the media, and executes it to perform the functions and elements of the invention described herein.

In the context of this description, the terms “user device,” “computer”, “smartphone,” and “tablet,” may be used herein interchangeably herein and refer to any electronic device capable of transmitting, receiving, displaying, outputting, inputting, and manipulating data. This includes, but is not limited to, mobile phones, personal digital assistants (PDAs), laptops, desktop computers, wearable devices such as smartwatches and fitness trackers, smart glasses, e-readers, gaming consoles, embedded systems in vehicles, smart home appliances, and any other computing devices that facilitate communication and data processing functions. These user devices are equipped with hardware and/or software components that enable them to communicate with databases and other servers over various network infrastructures. They can connect through any data network such the cloud or the internet, dedicated satellite links or any other wireless data network, utilizing wireless communication protocols such as any radio-frequency link, Wi-Fi, Bluetooth, and Near Field Communication (NFC). They may also use cellular networks like 3G, 4G, 5G, and/or 6G for data transmission. Additionally, these devices can establish connections via wired communication systems, including Ethernet networks, USB interfaces, or other wired networks.

The following non-limiting embodiment discloses a method and respective user(s) interactions of the invention in relation to one or more geotechnical and construction materials tests. Generally, the process begins with a project stakeholder submitting a request to perform one or more material tests. The request may be submitted by a project stakeholder via telephone, emailing, or an electronic request through the Portal. If a formal request is submitted via a phone call or email, an office user (e.g., a dispatcher) acknowledges the phone call/email and logs the request using the Portal. If an electronic request was submitted, an office user acknowledges the request in the Portal and the platform automatically logs it under a “Waiting for Approval” section of the Portal. However, in some scenarios there may not be a need for a formal request because one or more geotechnical and construction materials tests may be within the ordinary scope of a particular project and may be automatically populated.

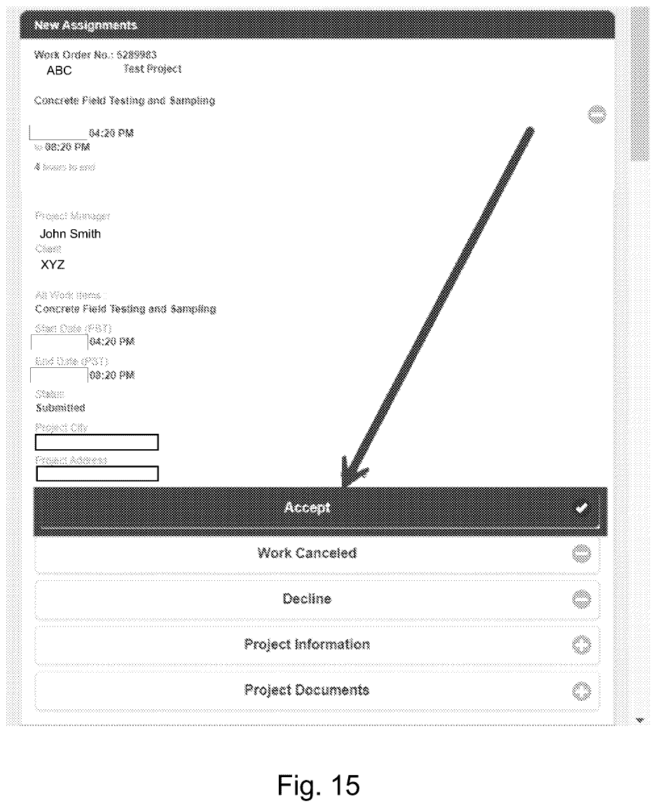

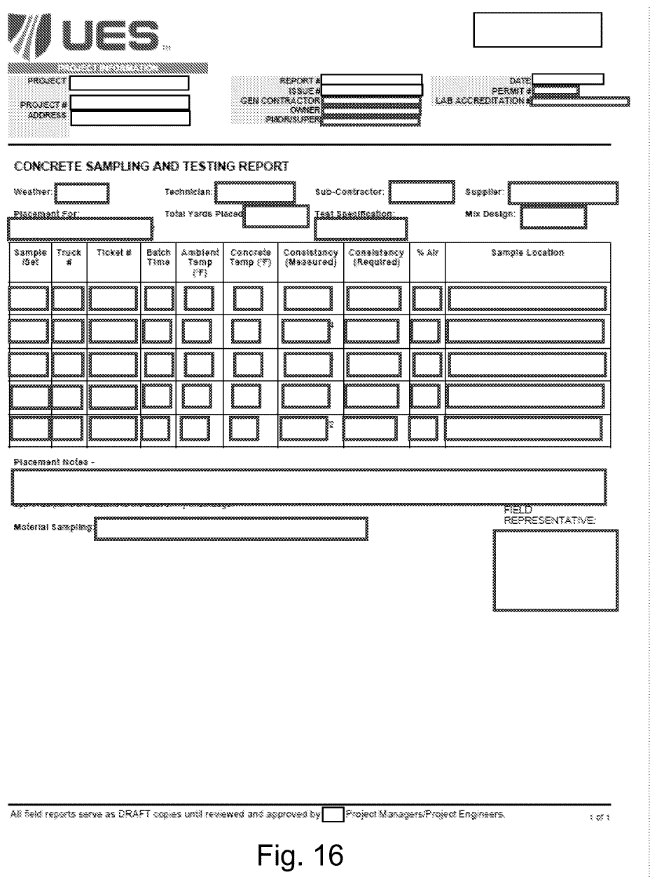

Generally, embodiments comprise a platform having a portal, wherein the portal comprises a graphical user interface for displaying project information and communication tools. In embodiments, the method comprising creating a work order via the Portal S101; accepting said work order via the portal 102 (e.g., see FIG. 15); performing one or more tasks associated with said work order S103; performing one or more tests associated with said work order S104; generating one or more reports S105 (e.g., See FIG. 16); and delivering said one or more report(s) S106.

In embodiments, the one or more tests relate to concrete testing and analysis, as described below.

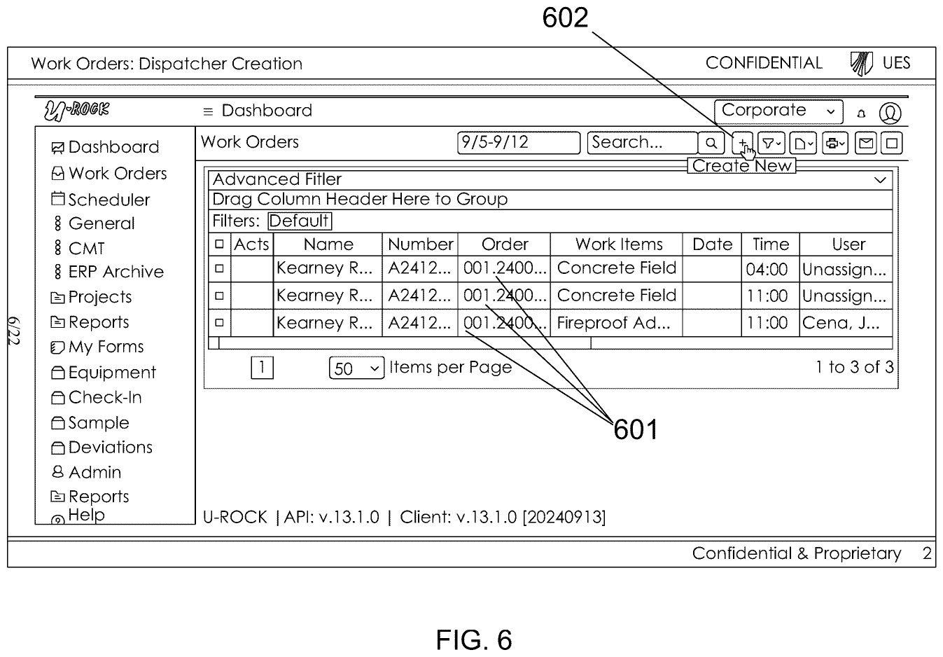

In Step S201 a User of the system may login to the platform using a portal to create a work order (WO). For example, and not intended to be limiting, a work Order may be a “Concrete Work Order” (CWO) and/or a “Field Density Work Order” (FDWO). Creation of a work order is typically done by an office user, field user, or a client user. Creation of a work order may be performed using a “scheduling” section of the Portal. A user navigates to the scheduling section of the portal and clicks a “Create Work Order” button 602 (See FIG. 6).

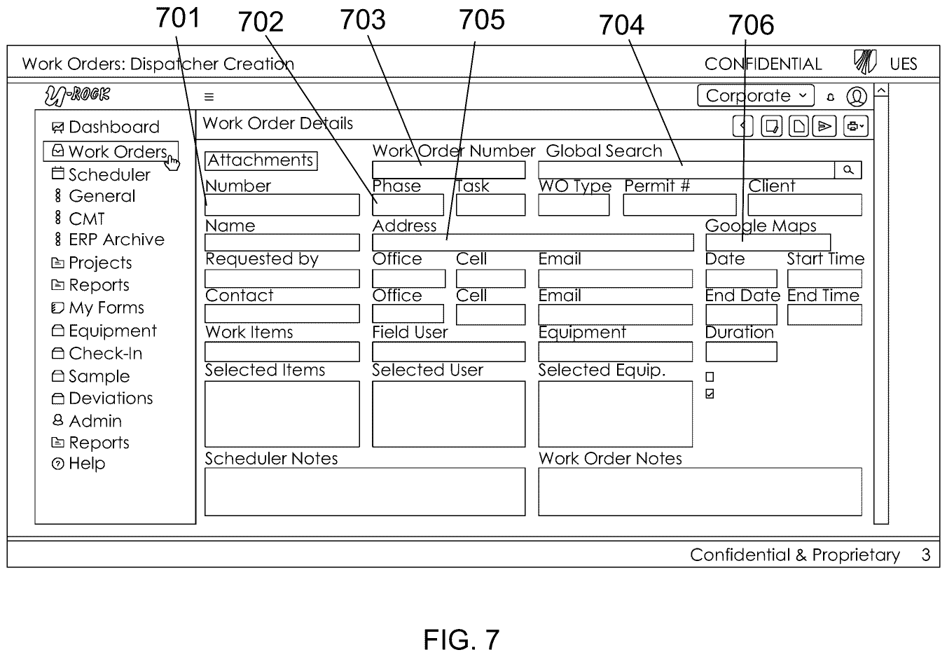

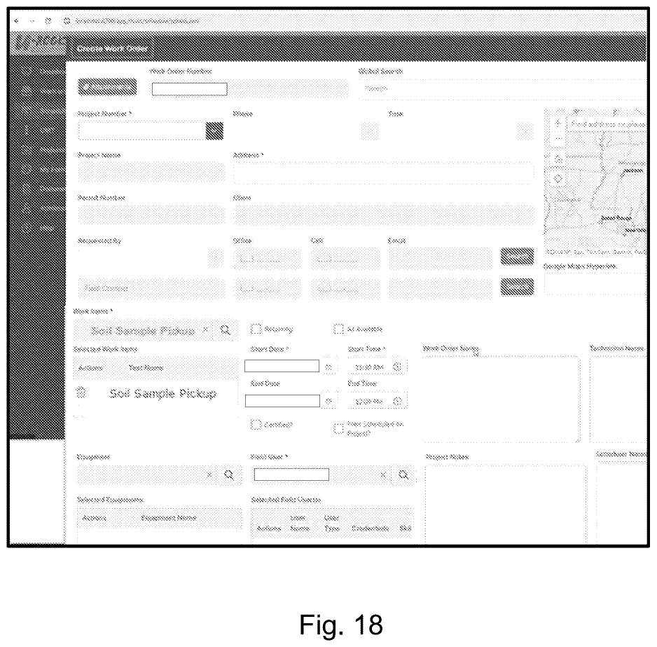

Referring to FIG. 7, the user enters all available information pertaining to the WO and saves it, if any required fields are left blank then the user may only save the WO as a draft. If the WO is only saved as a draft, the WO will sit under a “Draft WOs” section until the user goes back, completes all the required information, and saves it (See also FIG. 18).

During creation of the WO, the user may select a particular task and a work order number populates automatically and sequentially from a database and is maintained throughout the process.

During WO creation, the user may select and assign the WO to one or more qualified field users. The user may enter a starting date/time and an ending date/time. The user may navigate to the calendar schedule to view the WOs visually. The platform may then generate a notification of the WO to the assigned field user (e.g., a technician). The field user may receive a “push” notification, email, and/or a text message notification on their device indicating the assignment of the WO to them.

Next, in step S202, the field user assigned to the WO in S201 may acknowledge and accept the WO. In embodiments, WOs created in S201 may not be assigned to particular field users. In that scenario, the WOs may be viewable by one or more field users and allow a field user to accept a WO if they possess the requisite qualifications. Upon acceptance of a WO, the platform automatically sends the user who created the work order in step S201 a notification that the field user has viewed and accepted the work order.

Upon acceptance of the WO in S202, the method proceeds to one of steps S203 or S204 depending on the type of WO.

If the WO created in S201 is for a geotechnical and construction materials test and/or observation that occurs on-site which does not require sample collection, operation proceeds to step S203. For example, but not meant to be limiting, a geotechnical or construction materials test may be a concrete test or a field density test.

If the WO created in S201 requires geotechnical or construction materials testing, observation and/or a sample collection, operation proceeds to step S204.

If operation proceeds to step S203, the field user who accepted the WO in step S202 goes to the project site and performs one or more tasks associated with the WO. For example, tasks associated with a WO may be an on-site observation and/or field tests on concrete at the project site. In embodiments, the field user would take photographs and record notes of the site and upload the photos and captured notes using the portal. The field user's uploaded information would be saved in a database of the platform and also on the field user's user device as an offline backup.

In embodiments, the field user may generate a draft report at the site and may upload the report to the platform using the Portal, email the report, and/or print the report onsite.

In embodiments, the field user (e.g., technician) sends the draft report to a project administrator for initial review. The project administrator may then review the draft report and send the reviewed report to a second field user (e.g., a project manager). The second field user may then approve and sign-off on the field report. After the report is signed-off, the second field user (e.g., project manager) or another field user may then send the report to the client. The report may be sent to the client using the portal, email, or any other acceptable means.

If operation proceeds to step S204, the field user who accepted the WO in step S202 goes to the project site and performs one or more tasks associated with the WO. For example, tasks associated with a WO may be an on-site observation and/or field tests requiring one or more sample(s) to be collected. In embodiments, one or more samples may require casting concrete in cylinders or beams and leaving the one or more sample(s) on site to be picked up at a later date.

In embodiments, the field user in step S204 may use the portal to verify the field testing equipment is within calibration. The one or more tasks associated with WOs in step S204 may further require the field user to capture field data (e.g., notes and/or photos) for the project. In embodiments, the field user may use the portal to log the specimens collected (e.g., cylinders or beams) and record where the specimens are stored on-site. Recording where the specimens are stored on-site may further include uploading photos and/or inputting geolocation information (e.g., a drop-pin on a map) to the portal. If no internet connection is available, the portal will save the information offline on a user device and will auto sync to the portal when a network connection is re-established.

In embodiments, the first field user may also generate a draft report while on-site and upload the draft report using the portal. The first field user may then use the portal to transmit the draft report to a second field user (e.g. project administrator) for initial review. In some cases, the first or second field user may email or print the draft report on-site. The second field user (i.e., project administrator) may then review the draft report and send the reviewed report to a third field user (e.g., a project manager). The project manager may then approve and sign-off on the field report. Lastly, the second or third field user (e.g., project manager or project administrator) may then send the report to the client. The report may be sent to the client using the portal, email, or any other acceptable means.

In some scenarios, the first field user may be late to turn in their report associated with a WO. A report may be late when a field user hasn't logged their report in the portal prior to an office user submitting a work schedule for a future day. In which case, operation proceeds to step S205, wherein the portal will alert the first field user and display a window to help the first field user quickly log their report. The alert to the first field user notifies the first field user to enter their report and gives an option to enter specimen information, for example and not meant to be limiting, whether the specimens are cylinders or beams and where the specimen(s) are stored onsite. This information may include photos and/or geolocation information (e.g., drop-pin on a map).

Notwithstanding the above scenarios, upon transmission of the draft report to a second field user via the portal, operation proceeds to step S206. In S206 the portal automatically creates a second WO requiring a pickup of the specimen(s) collected in S204. In embodiments, the second WO may be automatically assigned to a field user. In embodiments, the second WO may not automatically be assigned to a field user, in which case the second WO sits in the unassigned WO queue of the Portal. If the second WO is unassigned, an office user may later assign the second WO to a field user. Alternatively, a field user may proactively view unassigned WOs and accept an unassigned WO they are qualified to accept.

Upon acceptance by a field user of the second WO created in S206, operation proceeds to step S207, wherein one or more specimens collected on-site must be picked up and transported to a laboratory for one or more tests to be performed on the samples.

In step S207, the field user who accepted the second WO travels to the site to pick up the one or more collected specimen(s). The field user may use the portal to view information associated with the second WO to find the one or more specimen(s) at the project site. Using the second WO information, the field user may find the one or more specimen(s), confirm the one or more specimen(s) match second WO information (location, number of specimens, specimen IDs, cast date), collect the one or more specimen(s), and load the one or more specimens in a vehicle for transport to a lab. The field user logs the successful pick-up of the one or more specimens using the portal. The field user may then transport the specimen(s) to the lab and drop the one or more specimen(s) in a designated area.

In some scenarios, the one or more specimen(s) collected in step S204 may become lost or destroyed between specimen collection and specimen pickup. In which case, operation proceeds to step S208. Using the second WO information, the field user attempts to find the one or more specimen(s) at the project site, but is unable to find them. The field user would then use the portal to mark the one or more specimens as “lost” or “destroyed”. The portal would then automatically notify an office user and/or a second field user (e.g. project manager). At which point, the portal would require either the office user and/or the second field user (e.g. project manager) to provide approval before the first field user leaves the site. Once the office or field user provides approval, the first field user is notified via the portal it is ok to leave the project site.

If pickup of the one or more specimen(s) is successful at step S207, operation proceeds to step S209. In step S209, the one or more specimen(s) are delivered and checked-in to an appropriate lab. The field user will use the portal and navigate to a “Specimen Check-in” section of the portal. The field user will match the specimen(s) in their possession with the appropriate specimen displayed in the portal. The field user can select the appropriate specimen in the portal and select “check-in”. The portal will display specimen information, for example and not limited to, sample(s) ID and cast date. In embodiments, the portal may also display the number of specimens, test age, test date (test date=cast date+test age), type (e.g., blank, field cure, hold), and/or condition (e.g., blank, frozen, damaged). A field user checking in one or more specimens at a lab may also use the portal to select the particular lab where the specimens are being left. The platform will automatically populate the time and date of check-in upon completion of the check-in by the field user.

Following completion of check-in at step S209, the method proceeds to step S210. In S210, the platform will automatically send a notification to an office user (e.g. dispatcher) and/or a lab user (e.g. lab manager). The notification indicating that the specimen has been checked-in to the lab and is ready to be received by a lab user.

After notification of check-in is transmitted, the method proceeds to step S211, wherein the one or more specimens are received by a lab user. In S211, one or more lab users are responsible for stripping specimens from their mold, transferring label from mold to specimen and ultimately storing specimens for future testing. A lab user may use the portal to navigate to a “Receive” section. In embodiments, the lab user may filter the received specimens by material type. For example and not mean to be limiting, the material type may be concrete, asphalt, or soil. The portal displays specimens checked-in by one or more field user(s). The specimens may be grouped by project and sorted by latest checked-in time & date. For each specimen, the portal may display a sample ID, specimen ID, test age, test date, project name, and the name of the user who checked in the specimen (e.g., field user). The lab user who “receives” a specimen strips the specimen from its mold and may write on each specimen the sample ID, specimen ID, test age, and test date.

The lab user uses the portal to check the specimen in as received. If there are any specimen(s) in the lab receiving area but not in the portal's “To Receive” section, a lab user can instantly search specimen information by entering the specimen ID into the portal. Upon clicking a search button, the portal may display one or more of the following: specimen ID, name of the person who cast the specimen (e.g., field user), a button to email this field user, cast date, name of the user who scheduled the specimen pick-up (e.g., office user), a button to email this user, name of the user who picked up the specimen, (e.g., field user), and a button to email this user. A key feature of the invention is that the specimen ID's are tied to the project WO number and unique identifiers are maintained throughout the process thereby providing an auditable chain of custody for a specimen.

After the lab user uses the portal to check the specimens as “Received”, the platform stores specimen information and auto populates lab testing queues for the scheduled testing dates, which may be based on “break” dates of the specimen. In embodiments, the lab user stores specimens in a curing room according to their break date. The platform automatically notifies an office user and a field user (e.g. project manager) that the specimens have been received and are stored in a curing room.

Next, operation of the method proceeds to step S212, wherein a lab user tests one or more specimens. In embodiments, the lab user will pull a specimen for testing and conduct a concrete cylinder strength test to assess the compressive strength of a hardened concrete specimen. A lab user may use the portal to view a testing queue of specimens that are awaiting to be tested. When today's date matches a specimen test date, the platform will queue those specimens in a “To Test” queue which is viewable using the portal. If specimens are being tested late, the platform may flag them in red. A lab user navigates the portal to the specimens “To Test” page wherein a list of specimens ready to be tested is displayed. In embodiments, the page may display a screen for bulk test data entry. A lab user enters test data, saves it and flags tests as completed. The platform stores test data and moves the next work queue as locally configured. In embodiments, the lab user performing the testing may select an option for a second lab user (e.g. lab manager) and/or a field user (e.g. project manager) to review the test. The portal allows a lab user to create draft lab test report to distribute internally or externally.

After testing of the specimen(s) is completed in S212, operation proceeds to at least one of steps S213 and/or S215.

In S213, a field user may view the lab report and complete a field report. The field user may submit their report using the portal. A second field user (e.g. project administrator) may make sure WOs assigned to field testing have reports submitted. The second field user may review all WOs for a particular date and search for WOs assigned to field testing & sampling. The second field user may contact first field user(s) who have not submitted their field reports. In embodiments, the second field user may also contact first field user(s) to complete their report if their report has missing information. The portal functions as a communication tool for second field user(s) to send email(s) with link(s) to WO(s) to first field user(s) to complete/correct their report. The second field user will review and check every field report to ensure information is complete. In embodiments, a second field user may make minor corrections to reports generated by first field user(s). If the report was signed-off by a first field user, the platform will store the signed version and create a new version for a second field user to make edits. If the report was signed-off by a first field user and corrections are done by the same first field user, the portal may ask the first field user if they would like to keep current version or create a new version. In embodiments, the platform will store the signed version and create a new version for a second field user to make edits. If a new version is created it will be noted on the report. The portal enables a field user to review a single report and/or also review bulk reports. The second field user (e.g. project administrator) may do a cursory review of each report before moving the report for a final review by a third field user (e.g. project manager). In addition, the portal allows field user(s) to communicate with an office user if any specimens were marked as cast in any of the field reports.

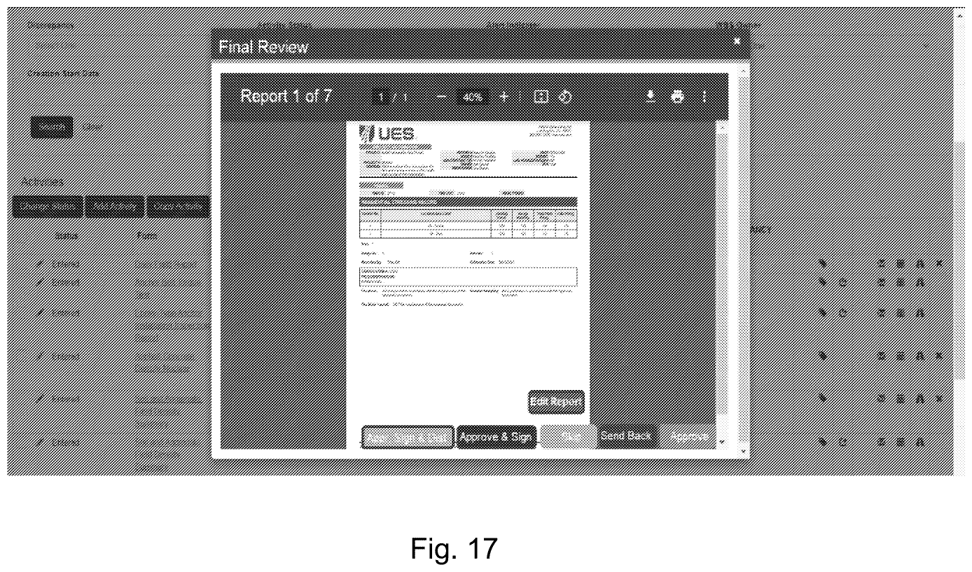

After initial review by a second field user in S213, operation proceeds to step S214 wherein a third field user (e.g. project manager) may perform final review of the report(s) (See FIG. 17). In S214, a third field user receives notification, via the portal, that one or more reports are ready for final review. In embodiments, some reports may be flagged as needing special attention and/or that the test results failed (i.e., the tested material did not meet specification). A third field user uses the portal to open each report and perform a technical review. The third field user may send incorrect or incomplete reports back to a second or first field user via the Portal (or email) with a link to a WO & report. If only minor corrections needed, a third field user may make the corrections. If the report was signed by a first or second field user, the portal will store the signed version and create a new version for the third field user to make edits. If the report was signed by a second or third field user and corrections are done by a second or third field user, the portal will ask the user if they would like to keep current version or create a new version. Any new versions are noted on the report. A third field user may eventually approve the reports, and associated billing, and timesheets. In embodiments, the portal allows a third field user to sign and/or seal the report(s). The third field user may send approved reports to a client user via the portal. In embodiments, the third field user may send approved reports to a second field user to distribute to a client user.

In embodiments, after completion of specimen(s) testing by a first lab user in S212, the lab test report undergoes review in step S215, wherein a first lab user is responsible for the initial review. In other embodiments, a second lab user (e.g. lab manager) is responsible for the initial review. The portal notifies a lab user that the lab reports are ready for initial review. The lab user undertaking responsibility for the initial review uses the portal to select one or more lab reports to review. In embodiments, a second lab user may send the lab report back to a first lab user to complete or correct the lab reports. In embodiments, a second lab user may perform the initial review and approve the lab report. Approving the lab report in the portal moves the lab report the final review queue. A second lab user (e.g. lab manager) may then perform final review of the lab report and approve the lab report and associated billing. Thereafter, the second lab user may use the portal to distribute the report to a second field user (e.g. project administrator), whereby the second field user may distribute the report to a customer user.

After initial lab report review in S215, operation proceeds to step S216 for final review of the lab report by a third field user (e.g. project manager). The portal automatically notifies a third field user of reports ready for final review. The third field user may select one or more reports to review. In the case of needing corrections, the third field user may make corrections or send the report back to a second lab user for corrections. The third field user may use the portal to approve the report and associated billing. Upon approval, the portal may transfer billing data to a third-party system (e.g. VantagePoint). The third field user may distribute the final approved report or may move the report to a second field user to distribute to the client user.

Lastly, delivery of any of the above-mentioned reports is performed in step S217. After any report has gone through final review, the report(s) will be placed in the “Report Delivery” queue of the portal. This function is typically carried out by a second field user but in some case a third field user or lab user may perform the distribution of a report(s). The particular user responsible for this task will be notified, via the portal, of reports pending for distribution. Reports can be distributed to a customer via email or the portal. This may be configured per project with the default being distribution to the customer via the portal. The user responsible for reporting will navigate to a report distribution page of the portal and select one or more reports and click a “Distribute” button. The portal will publish reports to a customer portal or email report(s), depending on a project report distribution configuration.

The foregoing description outlines one exemplary embodiment of the invention, providing illustrative detail to enable those skilled in the art to practice the invention. However, it should be understood that the scope of the invention is not limited to this embodiment. As will be further described below, alternative embodiments and variations of the method are provided that are also within the scope of the claims. These alternative embodiments may include modifications, additional steps, or variations in the sequence of steps that achieve similar results or provide complementary functionalities. Such alternative methods are disclosed to further expound the breadth of the invention, without imposing a restriction to any single embodiment or variation thereof.

In embodiments, field users, lab users, and office users may use the platform to more efficiently perform their job duties related to field density testing.

Operation begins at step S301, wherein a user of the system may login to the Portal to create a work order (WO). Creation of a work order is typically done by an office user, field user, or a client user. Creation of a work order may be performed using a “Scheduling” section of the Portal. The user creating the WO navigates to the “Scheduling” section of the Portal and selects a “Create Work Order” button. The user creating a WO uses the Portal to enter all available information pertaining to the WO and saves it. If any required fields are left blank during creation of the WO, then the user may only save the WO as a draft. If the WO is only saved as a draft, the WO will sit under a “Draft WOs” section of the Portal until the user goes back to the WO, completes required information, and saves the WO. Required information may be, but is not limited to, a task and/or a particular phase.

During creation of the WO, the user selects a particular phase and/or one or more task(s) and a work order number populates automatically and sequentially from a database of the platform. The work order number is maintained throughout the process. The platform will number each WO sequentially as they are created by a user. In embodiments, the WO number will be only used internally by the platform and the WO number will not be transferred to third-party systems. In embodiments, the WO number comprises a branch number, 2-digit year, and a 6-digit sequential number generated by the system. For example, and not meant to be limiting, a WO number may be 104.24315077. “104” corresponding to a Branch Number, “24” corresponding to the 2-digit year, and “315077” corresponding to the 6-digit sequential number. In embodiments, the WO number may further comprise a prefix to the sequential numbering indicating the type of report associated with the WO. For example, and not meant to be limiting, a WO requiring a Field Density Report may have “FDR” incorporated into the WO number (e.g., 104.FDR.24315077).

During WO creation, the user creating the WO may view a list of a plurality of field users wherein each field user of the plurality of field users has indications of availability and qualifications. The user creating the WO may view field users, select a field user, and assign the WO to the field user. The user creating the WO may enter a time window having a starting date/time and an ending date/time to complete items associated with the WO. In embodiments, the user may navigate to a calendar schedule view to see the scheduled WOs visually. The user may submit the WO via the Portal.

Upon submittal of the WO, the system generates one or more notifications of the WO to the assigned field user (e.g., a technician). The field user may receive a “push” notification, email, and/or a text message notification on their user device indicating the assignment of the WO to him/her. The field user may acknowledge and accept the WO which then causes the platform to send a notification back to the user who created the WO indicating that the field user has viewed and accepted the WO. The field user who accepts the WO may be referenced below as a first field user.

Once the first field user has accepted the WO, operation proceeds to step S302, wherein the first field user goes to the project site and performs one or more tasks associated with the WO created in S301. One or more tasks associated with a WO may be, but is not limited to, observing and reporting on the conditions at project site, collecting a soil sample, and/or testing a soil sample. A task requiring observing and reporting may comprise taking photographs and recording observations from the project site. These photos and observations may be uploaded through the portal to the platform. A task requiring collection of a soil sample comprises the first field user physically collecting the soil sample and using the Portal to log the collection of a soil sample and details of the soil sample. Non-limiting examples of soil details are: sample number, location details, specimen type and size, test assigned, sample date, and/or sample status. In embodiments, a field user may perform a field density test on-site.

Once one or more tasks associated with the WO are completed and logged using the Portal in S302, operation proceeds to step S303. In S303, the sample(s) collected in S302 are transported to the appropriate lab and checked-in. In embodiments, the first field user who collects and logs the sample(s) will transport the sample(s) to the lab for check-in. If the first field user is unable to check-in the sample(s), then any other user (e.g., a second field user, office user, or a contractor) may travel to the site to collect the sample(s) and transport them to a lab. In some cases, a field user logging collection of samples may come to the conclusion that the sample requires a new test. This is a key aspect of the invention, the ability of a user to use the Portal to “call back” a sample and add additional tests. As additional tests are performed on a sample, users are able to view the sample in the Portal to see all the previous tests that have been performed, as well as the original lab that performed the test, and any other labs in case it was transferred. In embodiments, requesting an additional test requires approval from a lab user (e.g., lab manager) or a second field user (e.g., a project administrator or project manager).

In step S303, the one or more specimen(s) are delivered and checked-in to an appropriate lab. The field user delivering the samples to the lab uses the Portal an navigate to the “Specimen Check-in” section of the Portal. The field user will match the specimen(s) in their possession with the appropriate specimen displayed in the Portal. The field user can select the appropriate specimen in the Portal and select “check-in”, resulting in a status of the specimen changing to “Delivered”. A user checking in one or more specimens at a lab may also use the Portal to select the particular lab that the specimens are being delivered to. The Portal will automatically populate the time and date of check-in upon completion of the check-in by the field user.

Following completion of check-in of the sample(s) at the lab (S303), the method proceeds to step S304. In S304, a lab user (e.g., lab administrator or lab manager) receives the sample and logs receipt of the sample in the Portal. Logging receipt of the sample changes the status of the sample from “Delivered” to “Received”. Once a sample is in “Received” status, the platform will move it to either an “Assign Test” status if no test is assigned, “Test Today” status, or “Future Test” according to what day testing was selected during Test Assignment.

In some cases, different tests for the same sample could be assigned to different lab users or transferred to different lab locations. The platform provides tracking and sample transferring by identifying which lab performed which test for tracking and billing purposes. Inter-Lab charges are tracked by the platform when sample testing is performed by other labs. In embodiments, a sample ID may have multiple tests so the samples may further comprise a test ID in case they are moved from one lab to another. In the case of moving samples to different labs, only a lab manager or project manager can approve.

In any case, sample(s) received in S304 undergo testing in step S305. In S305, a first lab user may pull a soil sample and perform one or more tests on the soil density and/or moisture content of the soil

Upon completion of testing in S305, operation proceeds to step S306 wherein the first lab user generates a proctor report. A “proctor report” is the laboratory compaction test report for a specific soil sample that establishes the soil's reference compaction curve, including the maximum dry density and the corresponding optimum moisture content for that soil under a defined compactive effort. These reference values are produced in the lab and then recorded in the report so they can be used as the basis for field acceptance criteria. The proctor report is logged using the portal wherein a second lab user (e.g., lab manager) or a second field user (e.g., project manager) can access and review the proctor report. In embodiments, a second lab or field user (e.g., lab manager, project manager) may review the proctor report. If a second lab user approves the proctor report then the proctor number with its values can officially be used. Approval of the proctor report is logged by the platform and accessible by field users via the portal. Once the proctor report is approved, operation proceeds to step S307.

In S307, a field user uses the proctor report to perform one or more field density tests at a project site. Some non-limiting examples of field tests that may be performed are: drive sleeve, nuclear gauge, sand cone, e-gauge, moisture, check point, and ballon test. Field users may use the portal to change the requirement of moisture (+/−) by location or by test. A failed test may be logged using the portal and may require re-testing.

In any of the above embodiments, one or more tests performed by a field user or lab user may “fail”. That is to say, the results of the test may exceed an upper predefined threshold or may not meet a lower predefined threshold. For example, a concrete strength test on a cylinder of concrete may reveal that the concrete is unable to withstand a requisite pressure or a field density test may review that moisture content is too high or the soil needs addition of aggregate.

In embodiments, the system employs a server architecture where one or more servers 1401 comprise software to perform specific functions essential for efficient project management. The software residing on the one or more servers 1401 specializes in handling distinct aspects of project workflows, for example but not limited to, task allocation, resource management, time tracking, communication, and reporting. This specialization allows for enhanced performance and efficiency within each functional area, leading to a more streamlined and effective overall system.

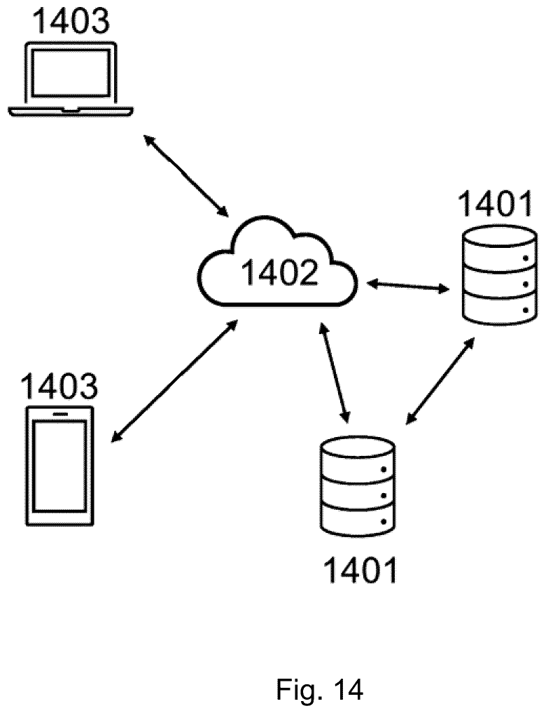

Referring to FIG. 14, one or more servers 1401 may interact with one or more user devices 1403 over a data communication network 1402, enabling seamless communication and data exchange between them. The data communication network 1402 may comprise various technologies, including cloud services, the internet, wireless communication protocols like Wi-Fi and Bluetooth, cellular networks such as 4G, 5G, 6G, Ethernet, and other wired communication systems. By utilizing standard communication protocols and data formats, the one or more servers 1401 ensure interoperability and consistent data flow across the system. This interconnectedness allows for real-time updates and synchronization, ensuring that all components of the project management system are up-to-date.

In embodiments, the software may be integrated into a single server or distributed among a plurality of servers. In a single-server configuration, the software may reside on one physical or virtual server, simplifying deployment and maintenance while providing efficient intra-system communication. This setup is often suitable for smaller organizations or projects with limited scope, where simplicity and cost-effectiveness are priorities. In embodiments, a distributed server architecture may be employed where one or more servers may be located in different geographical locations or within cloud environments. This distribution enhances scalability, load balancing, and redundancy, ensuring that the system can handle increased workloads and maintain availability. By supporting both single and distributed configurations, the system can be tailored to meet the specific needs and constraints of various organizations.

The one or more servers may be interconnected with various databases through a combination of synchronous and asynchronous communication methods over a network. RESTful APIs are used for standard request-response interactions between databases and the one or more servers.

The portal is a pivotal component of the platform, providing users with a secure and personalized interface to engage with project data. Through role-based access control, users are granted certain permissions, ensuring they receive relevant information without compromising data security. In embodiments, the portal may have one or more of the following functionalities:
i) allowing clients to access project documents, ii) receiving push notifications/alerts when reports are ready; iii) status updates on projects indicating any changes and/or milestones, iv) historical database of client's work, and v) ability to answer surveys on reports. These functions may be facilitated by automatic notifications that keep clients informed without manual intervention. Integrated communication tools within the portal enable direct messaging and feedback exchange between clients and project personnel, fostering a collaborative environment and enhancing client satisfaction.

This portal can adapt to cater to different client needs and technological environments, all while maintaining security standards and integration with other system modules.

In embodiments, the portal may be accessed via a dedicated application running on a user device 1403 or a web-based interface using standard web browsers. This interface employs responsive design principles to ensure compatibility across desktops, tablets, and mobile devices, providing clients with flexibility in how they access project information.

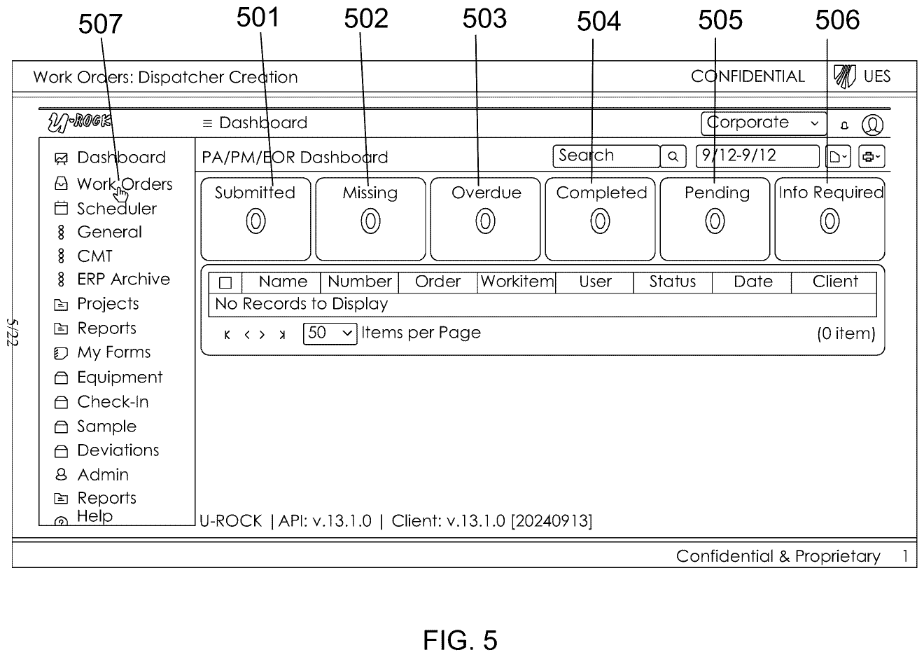

Referring to FIGS. 5-7, embodiments of a web-based user interface are illustrated. FIG. 5, may be a “home page” for a user of the portal. From this home page a user can get an overview of the status of their work orders. For example, but not meant to be limiting, a user can see the number of submitted work orders 501, work orders where there has been an indication that the sample is missing 502, work orders having an overdue task 503, completed work orders 504, pending work orders 505, and work orders requiring more information 506. Selecting the “work orders” button 507 in the left column may take you to a web-based interface shown in FIG. 6.

Referring to FIG. 6, a web-based user interface is shown which may allow a user to view existing work orders 601 and create new work order(s) 602. After selecting create new work order 602, a user may be directed to a web-based interface according to FIG. 7 for inputting work order details into their appropriate fields.

The work order details view may have multiple fields to capture, organize, and confirm entries necessary to instantiate and manage a work order. As discussed above, the work order details view may be accessed from within the broader home page dashboard (FIG. 5) to centralize data entry for identification, client, scheduling, resource, and documentation elements of the work order. The work order details interface provides fields for core work order identifiers, including number 701, phase 702, and work order number 703, enabling a user to specify or confirm the unique designations for the record at creation time or during subsequent edits. The view further includes a global search 704 control to assist the user in locating related records or reference data while entering work order information, supporting efficient cross-referencing during data entry. Task-related metadata may be entered through controls labeled Task, WO Type, and Permit #, allowing the user to capture the categorical work type and any permitting identifiers that may be associated with the contemplated activities.

Client-related data entry is supported through fields that identify the organizational or individual client and the specific contact name associated with the work order. Furthermore, the user may input an address 705, and an associated Google Maps control 706 is available to validate or visualize the location, facilitating accurate entry of the service site or project location. The interface may further expose contextual action or reference controls, allowing the user to annotate or link to enterprise records as part of the data entry workflow, thereby maintaining continuity with related systems or comments.

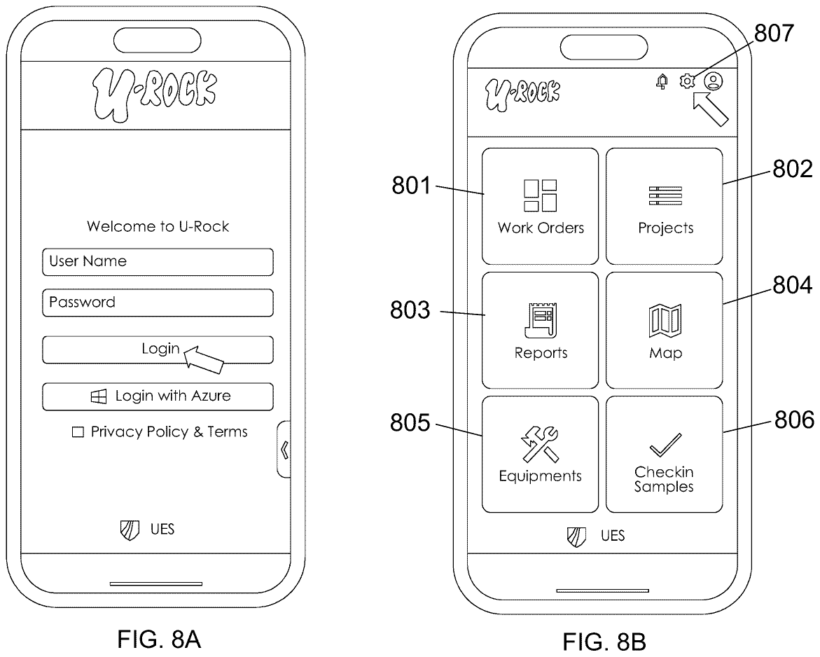

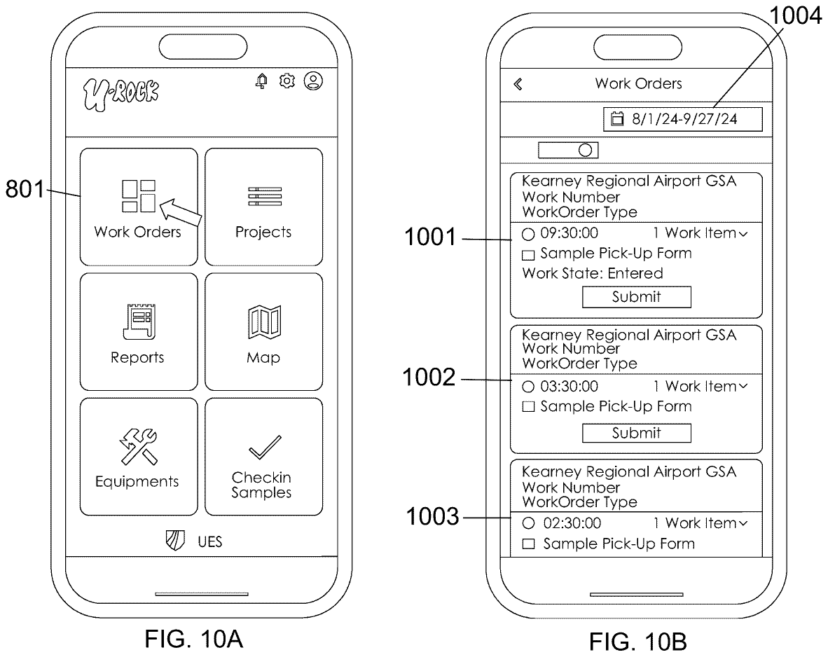

In embodiments, the invention can take the form of an application for a user device. Referring to FIGS. 8A-13B, mobile application user interfaces are illustrated. This structure leverages native mobile functionalities, such as push notifications and biometric authentication (e.g., fingerprint or facial recognition), to enhance security and user experience. The mobile app communicates with backend services via secure APIs and uses local storage with encryption for caching data when offline.

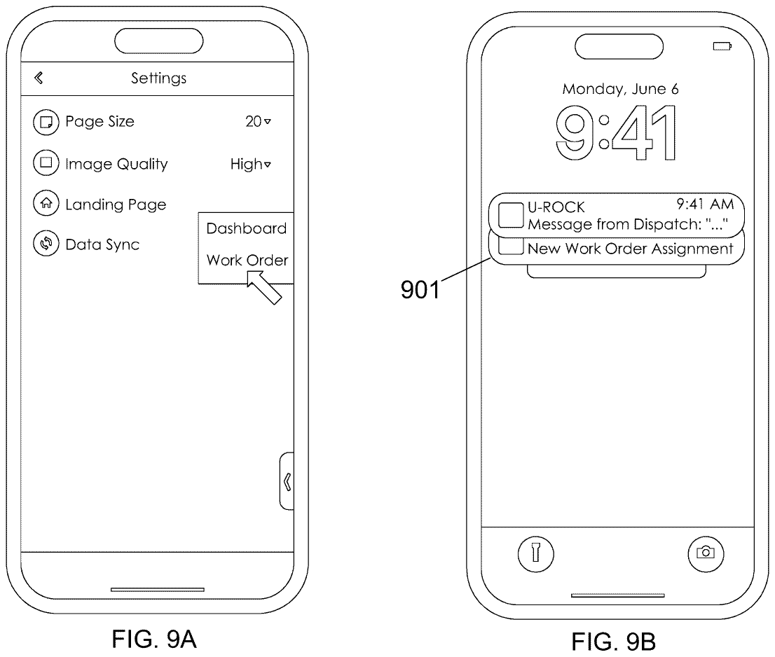

A mobile application interface provides field personnel with streamlined access to work order functions, beginning with an authentication screen FIG. 8A that accepts a User Name and Password and optionally supports Login with Azure, with links to Privacy Policy & Terms. Upon successful login, the user is presented with a mobile landing page FIG. 8B exposing primary modules, including Work Orders 801, Projects 802, Reports 803, Map 804, Equipments 805, Checkin Samples 806, enabling one-tap navigation among operational views optimized for on-site workflows. A user may also visit a settings panel shown in FIG. 9A by selecting the settings gear 807.

Referring to FIG. 9A, the settings panel allows configuration of Page Size, Image Quality, and a user-selectable Landing Page, and includes a Data Sync control for managing synchronization of application data with the backend service

Referring to FIG. 9B, the mobile application may transmit notification communications contextually; for example, a banner message 901 from Dispatch may be displayed in conjunction with a New Work Order Assignment, notifying the user of assignments and instructions as they are issued to the device.

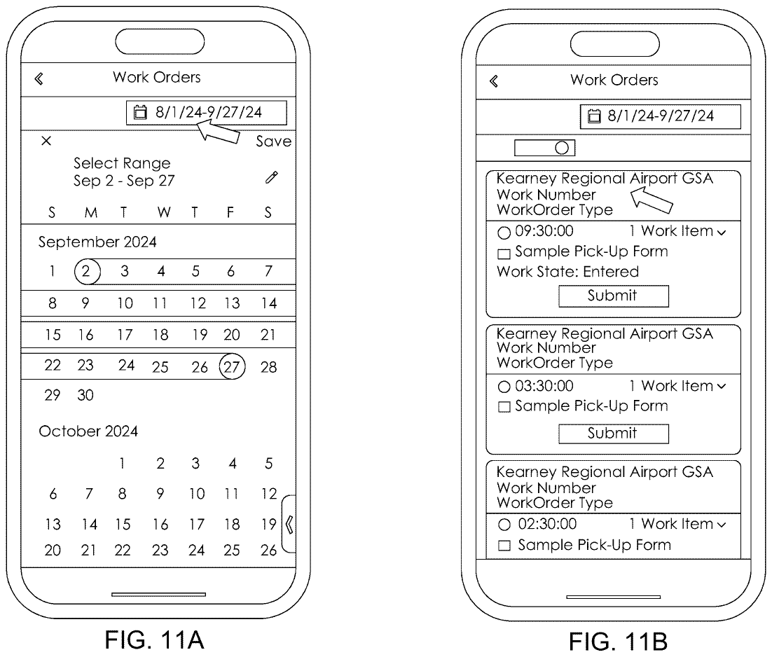

Selection of the Work Orders module 801, may present the user with a list view showing a date-scoped collection of items (e.g., Work Orders summary for a selected range) where each card 1001-1003 displays work order information and site identifiers such as Kearney Regional Airport, the scheduled time, the count of Work Item(s), and action affordances including Submit and a status indicator such as “Work State: Entered” for at-a-glance progression control. A date-range selector 1004 supports Select Range actions (see FIG. 11A) and renders a monthly calendar grid to filter the work list to relevant dates for the mobile user.

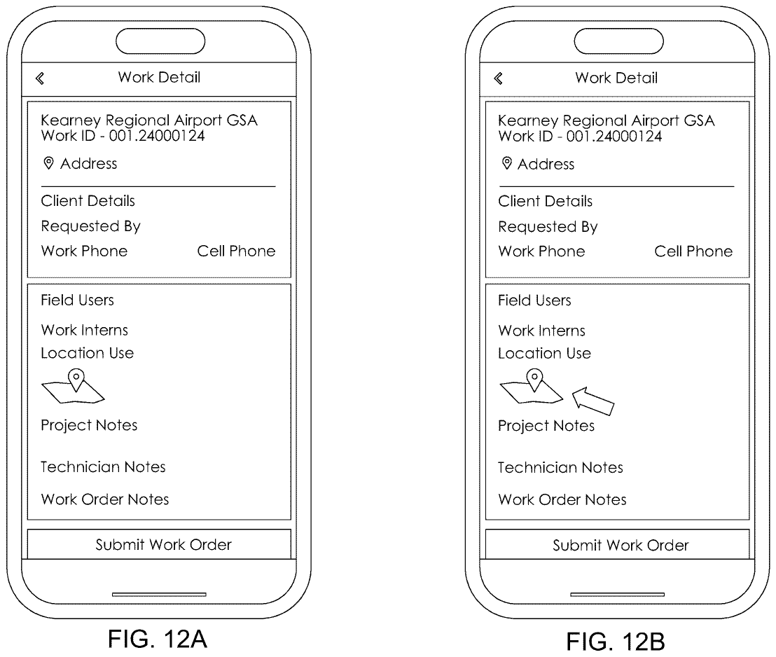

Selecting a list item, for example card 1001, transitions to a Work Detail view (see FIGS. 12A-12B) that consolidates editable fields appropriate for field execution, including but not limited to, Work ID, Address, Client Details, Requested By, Work Phone, Field Users, Work Interns, Location Use, Project Notes, Technician Notes, and Work Order Notes, concluding with a Submit Work Order control to finalize entry from the device.

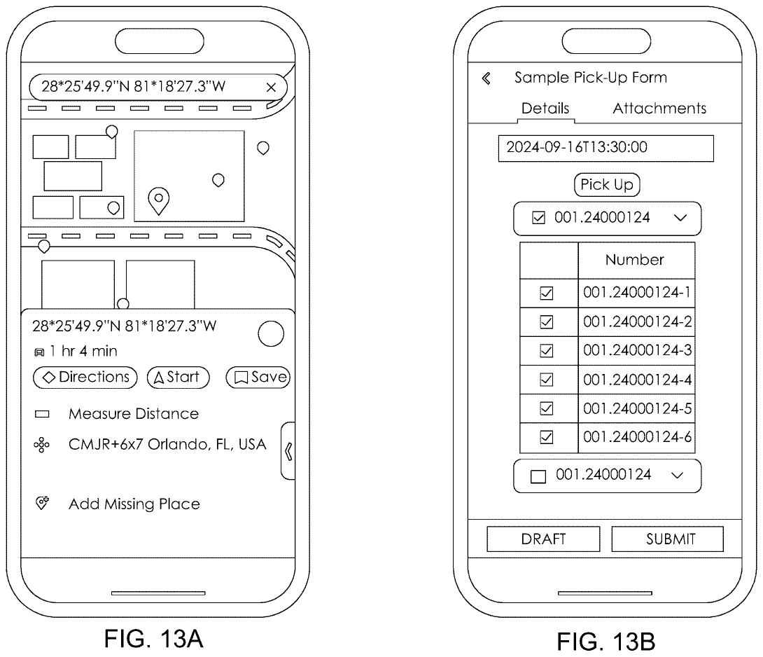

Location-aware functionality is provided through a mapping pane (see FIG. 13A) that displays geographic coordinates and supports Directions, Save, and Measure Distance functions, facilitating on-site navigation and verification of the service location. For task-specific data capture, a Sample Pick-Up Form (see FIG. 13B) exposes Details and Attachments views, presents a scheduled timestamp (e.g., 2024-09-16 T13:30:00) and task descriptor (Pick Up), lists associated sample numbers, and provides DRAFT and SUBMIT actions to manage state transitions as the field user completes the workflow.

In embodiments, the platform implements a hierarchical folder structure that normalizes content placement across projects, departments, and clients, thereby enabling consistent governance, access control, and automated processing. The folder structure may be created automatically upon the creation of a new project or proposal record in Vantagepoint.

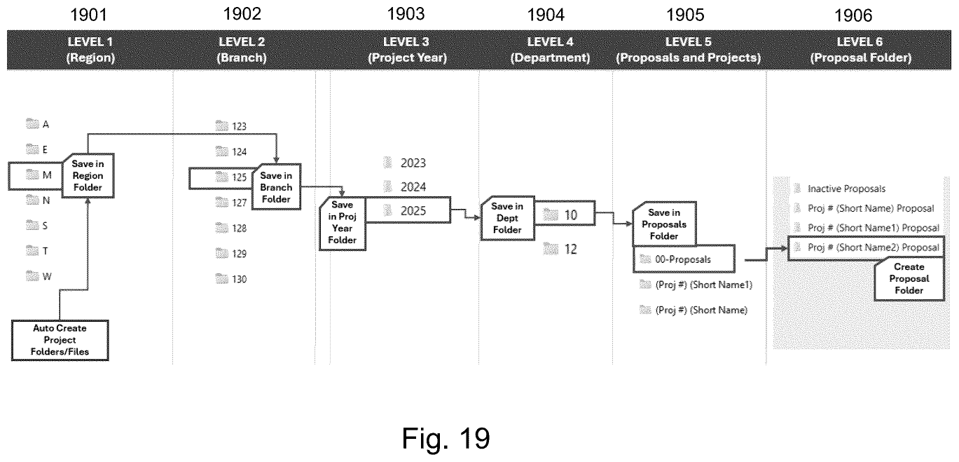

In embodiments, the file system hierarchy may be “Project-Focus,” meaning that organizational metadata culminates in a project-centric node prior to user-defined content. The hierarchy may comprise seven levels. Referring to FIG. 19, a structured, multi-level folder hierarchy configured to organize proposal content. The hierarchy comprises six discrete levels arranged in a parent-child relationship. The seven levels, from highest to lowest, may be as follows: Level 1—Region 1901; Level 2—Branch 1902; Level 3—Project Year 1903; Level 4—Department 1904; Level 5—Proposals and Projects 1905; Level 6—Proposal Folder 1906, and Level 7 (not shown)—User-Defined Folders, each of which is explicitly labeled in the figure to convey both structural order and intended use. In operation, the system is configured to auto-create one or more folders at designated levels based on discovered organizational information, which enables deterministic storage of records within an information management repository while reducing manual user intervention.

At Level 1 1901, folders are partitioned by region, providing a top-level segregation of content aligned to geographic or organizational territories, exemplified by alphanumeric or alphabetical region identifiers depicted across the figure (e.g., A through Z). Level 2 1902 further subdivides the region by branch, thereby narrowing the scope to a local operating unit or office that is associated with the parent region and used to direct downstream folder creation and placement. At Level 3 1903, the hierarchy is keyed by project year, which is shown by year-labeled folders such as 2023, 2024, and 2025; these year partitions serve as temporal containers for proposals and projects initiated, stored, or otherwise managed during the corresponding calendar period. Level 4 1904 introduces departmental categorization, where department-specific folders (e.g., “Dept 10,” “12”) provide function-based separation and enable department-driven retention and access policies downstream. Level 5 1905 aggregates proposal and project content under a “Proposals and Projects” node. Level 6 1906 may comprise active proposals and a distinct “Inactive Proposals” location for materials that have moved to a non-active state, thereby distinguishing in-flight proposal artifacts from archived or non-progressing records while maintaining their association to upper-level context (Region, Branch, Year, Department).

At Level 6 1906, proposal-specific folders may be identified by project number and short name, illustrated as “Proj #(Short Name),” “Proj #(Short Name1),” and “Proj #(Short Name2),” each of which corresponds to a distinct proposal. Proposal folders in Level 6 1906 may be created automatically by the system based on the system detecting or “finding” relevant organizational metadata and proposal identifiers from an upstream application (e.g., the Vantagepoint system), thereby ensuring that standardized folder names and structures are provisioned without manual setup. In an exemplary implementation, the system automatically determines the appropriate Region, Branch, Project Year, and Department from organizational data and creates a target path under Level 5 1905, then instantiates a proposal folder at Level 6 1906 with the corresponding project number and short name for consistent filing of proposal artifacts. Level 7 (not shown) may consist of user-defined folders.

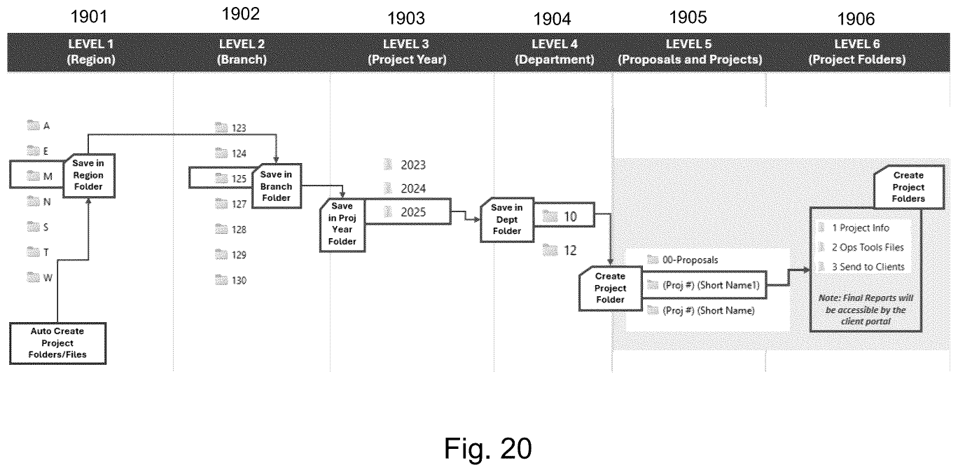

In embodiments, the file system hierarchy may be “Project-Focus,” meaning that organizational metadata culminates in a project-centric node prior to user-defined content. The hierarchy may comprise seven levels. Referring to FIG. 20 a structured, multi-level folder hierarchy configured to organize content for projects. In particular, the depicted embodiment corresponds to proposals designated as “Won,” such that the hierarchy illustrates placement following award of a proposal. In the “Won” configuration, these Level 6 folders operate as containers for the awarded work. Level 7 (not shown) may consist of user-defined folders.

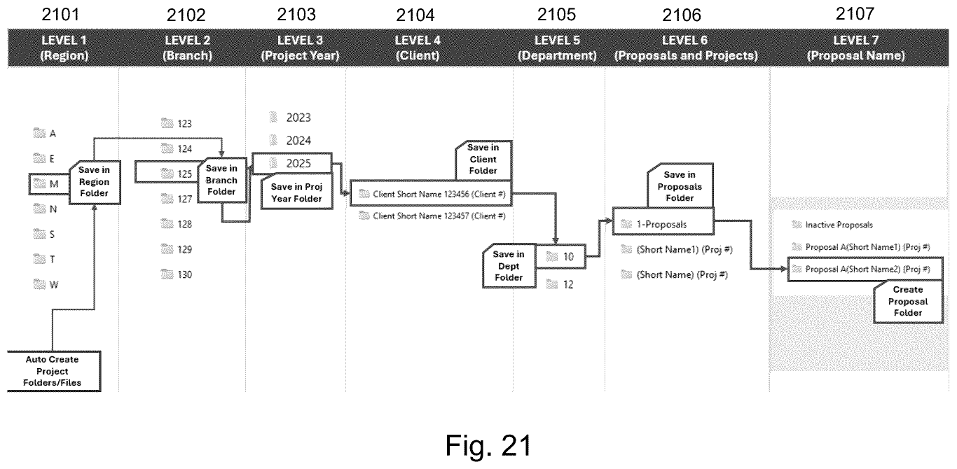

In embodiments, the file system hierarchy may be “Client-Focus,” emphasizing client identity as a primary organizing feature before departmental ownership. The hierarchy may comprise eight levels. Referring to FIG. 21, the hierarchy may organized into eight ordered levels: Level 1—2101; Level 2—2102; Level 3—Project Year 2103; Level 4—Client 2104; Level 5—2105; Level 6—Proposals and Projects 2106; Level 7—Proposal Name 2107, and Level 8 (not shown)—User-Defined Folders, thereby ensuring that each proposal is deterministically addressable via a unique path constructed from standardized organizational attributes.

This embodiment is similar to the embodiments shown in FIGS. 19 & 20, but Level 4 2104 is a client-designated container identified by a standardized client short name and, in certain implementations, an associated client identifier. For example, but not meant to be limiting client containers may appear as “Client Short Name 123456” or “Client Short Name 123457”. The system can automatically resolve and store proposals under the appropriate client container upon detecting organizational information sufficient to determine the correct storage path.

At Level 6 2106, the structure bifurcates or groups artifacts into proposal-centric and project-centric containers, which may be labeled as “1-Proposals,” or “(Short Name) (Proj #)” and related classifications.

At Level 7 2107, the system creates a proposal-specific container whose name is derived from elements such as client short name, proposal designation, and, where applicable, an internal project or proposal number. Level 8 (not shown) enables User-defined folders.

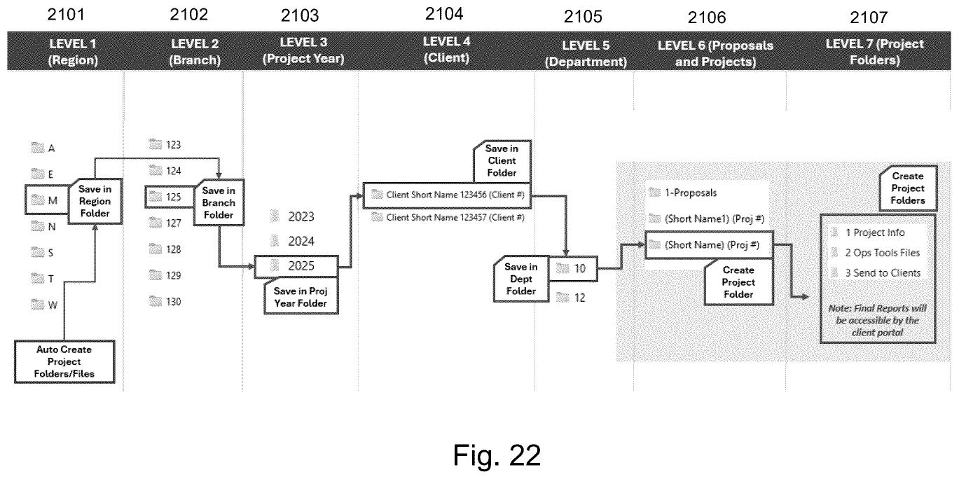

In embodiments, Referring to FIG. 22, a structured, multi-level folder hierarchy configured to organize content for projects. In particular, the depicted embodiment corresponds to proposals designated as “Won,” such that the hierarchy illustrates placement following award of a proposal. In the “Won” configuration, these Level 6 folders operate as containers for the awarded work. Level 7 may consist of folders such as, but not limited to, “1 Proj Info”, “2 Ops Tool Files”, and “3 Send to Client”. Level 8 (not shown) may consist of user defined folders.

Referring to FIGS. 20 & 22, in embodiments, anything in the “3 Send to Clients” Folder will be Accessible to Share with clients.

For clients requiring integration with their internal systems, the portal can provide an API integration layer. This structure allows clients to fetch and interact with project data.

Regardless of the structural form, the portal communicates over secure network connections, utilizing HTTPS protocols with TLS encryption to safeguard data in transit. The software may verify client credentials and with a role-based access control (RBAC) system to enforce permissions. A notification module may be used to provide real-time updates to facilitate communication between users.

To provide clients with a secure and personalized interface to engage with project data, the portal follows an algorithm comprising: i) a client authentication step wherein the portal accepts the client's username and password input through a secure login form. The password may be encrypted using a secure hashing algorithm. The platform may query an authentication database to compare the encrypted password with the stored hash. If the credentials match, it generates a session token using JSON Web Tokens (JWT) with embedded claims about the user's identity and role. The session token is sent to the client and stored securely, establishing a secure session for subsequent interactions. If the credentials don't match, the portal returns an authentication error.

During a role-based access control enforcement phase, the platform extracts a user's role and associated permissions. The portal may be rendered dynamically based on a user's permissions, ensuring the portal user only sees and interact with permitted features and data, thus personalizing the experience while maintaining security.

The portal delivers real-time updates on project milestones, changes, and progress through automatic notifications. This mechanism keeps clients informed without requiring manual updates from the project team, thereby enhancing transparency and trust. Notifications are configurable, allowing clients to select the types of updates they wish to receive and their preferred delivery methods, thereby personalizing their engagement with the platform.

By integrating role-based access control, the portal ensures that users receive information tailored to their needs without exposing sensitive data. Permissions are assigned to align with each user's role, granting access to relevant projects, documents, and communication channels while restricting unauthorized areas. This enhances security and provides a streamlined user experience, as users are not overwhelmed with unnecessary information.

The platform facilitates detailed resource allocation and assigning personnel/equipment where they are most needed. For example, but not limited to, enabling the capturing and/or fulfilling work orders, personnel/equipment scheduling, personnel/equipment assignments, personnel/equipment dispatching, tracking of work orders, samples, equipment management (availability, calibrations, location), and status communication to clients.

The platform empowers field personnel by equipping them with mobile applications capable of real-time data collection and analysis. This provides functionality for reporting & documentation, personnel time capture, and communication among stakeholders.

Furthermore, the mobile application that users interact with is designed with offline functionality, allowing data entry in environments without internet connectivity. Once connectivity is restored, data is automatically synchronized. Real-time data validation mechanisms may be in place to ensure accuracy and completeness at the point of entry, minimizing errors that could propagate through the system. The mobile applications support multimedia inputs, enabling users to attach photos, videos, and voice notes to specific data entries, enriching the data set with contextual information.

The platform tracks samples from collection through testing and reporting, providing transparency and traceability at each stage. The data collected by lab personnel may be pushed to a user device or available for viewing in the portal. Such data may include, but is not limited to, sample tracking & status (i.e., how many tests are complete), test calculations & analysis, reporting, sample disposal notifications, equipment utilization, scheduling and lab hour optimization.

Automated reporting features generate test reports and distribute them to relevant stakeholders without delay. The platform ensures that all testing procedures adhere to regulatory standards, with compliance management tools that monitor and enforce adherence to industry-specific regulations.

The platform tracks performance metrics, providing insights into progress against key performance indicators and project milestones, which aids in identifying areas that may require attention or adjustment.

The form and reporting capabilities of the platform allow organizations to standardize their documentation processes. Users can design custom templates that reflect organizational standards and requirements. The platform supports automated distribution of reports, scheduling them to be sent to stakeholders at predefined intervals or upon the completion of certain tasks. Data export functions enable users to extract data in various formats, facilitating external analysis or archival purposes.

The platform's database integration features ensure seamless data exchange with client and third-party systems. Through standardized Application Programming Interfaces (APIs), the platform can connect with external databases, allowing for real-time or scheduled data synchronization. Security protocols, including encryption and secure authentication methods, protect data during transfer, maintaining confidentiality and integrity.

In embodiments, the platform comprises an artificial intelligence (AI) module to perform predictive analytics, forecasting project timelines, resource requirements, and potential bottlenecks based on historical and real-time data. Anomaly detection capabilities identify unusual patterns or deviations that may indicate underlying issues, enabling proactive interventions. The AI module may provide decision support by offering recommendations derived from data trends, assisting users in making informed choices.

All these functionalities are unified within a comprehensive portal (i.e., a dashboard user interface) that serves as the central interface for users. The dashboard is highly customizable, allowing users to tailor the display to their preferences and focus on the metrics most relevant to their roles. Interactive elements enable users to drill down into data for in-depth exploration, while real-time updates ensure that decisions are based on the most current information available.

From a security and compliance standpoint, the platform is designed to meet the highest industry standards. Data encryption is employed both at rest and during transmission, utilizing robust encryption algorithms to protect sensitive information. Detailed access logs record user activities, providing an audit trail that supports accountability.

In terms of implementation, the platform leverages scalable cloud technologies that guarantee high availability and performance. Its architecture allows for modular deployment, making it easier to maintain and update individual components without impacting the entire system. The mobile applications are developed to be compatible with major operating systems, including iOS and Android, ensuring broad accessibility for users across different devices.

Each user device and server system may include one or more processors configured to execute program instructions and one or more memory components configured to store instructions and data. The processors may include general-purpose CPUs, GPUs, or other specialized processing units. The memory components may include volatile memory (e.g., RAM) and non-volatile memory (e.g., flash, ROM, NVRAM), and the systems may further include persistent storage devices such as solid-state drives, hard disk drives, optical media, or other non-volatile storage technologies. The systems may be provisioned, orchestrated, or managed using any suitable platform or framework, and may include container runtimes, orchestration layers, configuration management systems, identity and access management systems.

The software enabling the portal may be implemented as computer program instructions stored on one or more non-transitory computer-readable media. The non-transitory computer-readable media may include, by way of example and not limitation, magnetic storage media, optical storage media, magneto-optical storage media, flash memory, solid-state storage, or other tangible media configured to store instructions. The instructions, when executed by one or more processors of a computer or server system, a user device, or both, cause the processors to perform any of the operations, processes, methods, or functionalities described herein, including but not limited to creating and managing projects, assigning and tracking tasks, generating and updating schedules, allocating and re-allocating resources, generating reports, and facilitating collaboration between users. The term “non-transitory computer-readable medium” expressly excludes signals per se, carrier waves per se, and other transitory media. In certain embodiments, portions of the instructions may be implemented as firmware or code, and portions may be implemented in hardware, such as application-specific integrated circuits (ASICs), field-programmable gate arrays (FPGAs), or other logic devices.

In operation, the user devices may transmit user inputs, project data, task updates, files, and other content to the server systems via the data communication networks, and the server systems may process such data, update one or more data stores, and return output data including project state, task status, reports, analytics, notifications, or other computed results. The system may support synchronous and asynchronous communication models, including request/response, publish/subscribe, and message queuing. In some embodiments, all or a subset of processing may be performed on the user devices to support offline operation, with synchronization of state to the server systems upon data network availability.

In summary, the invention presents a holistic solution that addresses the multifaceted challenges faced by organizations in coordinating laboratory, office, field, and client operations. By integrating advanced technologies such as GIS and AI within a user-friendly and secure platform, it enhances operational efficiency, improves data accuracy, and fosters effective communication among all stakeholders.

Although the above description may contain specific details, they should not be construed as limiting the claims in any way. Other configurations of the described embodiments of the disclosed systems and methods are part of the scope of this disclosure.

It is understood that the embodiments described herein are exemplary and are provided to illustrate the principles of the invention. The methods, systems, and processes described above may be implemented in a variety of configurations and are not limited to the specific arrangements disclosed. Furthermore, the steps of the disclosed methods may be performed in any practical order, combined in various ways, or implemented individually without departing from the scope of the invention as defined by the appended claims. It is intended that the scope of the invention encompass all modifications, substitutions, and variations that fall within the spirit and broad scope of the claims, including equivalent structures and functionalities.

It will be appreciated that various of the above-disclosed and other features and functions, or alternatives thereof, may be desirably combined into many other different systems or applications. Also, various alternatives, modifications, variations or improvements therein may be subsequently made by those skilled in the art which are also intended to be encompassed by the following claims.

The flowcharts and block diagrams presented in the Figures are illustrative of the architecture, functionality, and operation of various embodiments of the systems, methods, and computer program products disclosed herein. Each block depicted in the flowcharts or block diagrams may correspond to a module, segment, or set of executable instructions configured to perform one or more specified logical functions. It should be understood that, in certain alternative embodiments, the order of functions as represented by the blocks may vary. For example, the execution of two successive blocks may occur substantially concurrently or in reverse order, depending on the specific functional requirements. Furthermore, each block or combination of blocks in the flowchart and/or block diagram illustrations described herein may be implemented, in whole or in part, on or within a non-transitory computer-readable medium. Such a medium may include instructions that, when executed by one or more processors, cause the processors to perform the functions, steps, and processes described above. Accordingly, the embodiments of the invention disclosed herein are not limited to any specific hardware or software configuration and may be realized in any suitable computing environment without departing from the scope and spirit of the invention as defined by the appended claims.

What is claimed is:

1. A method for providing multi-point connectivity among geotechnical or construction materials project stakeholders, the method comprising:
providing a portal having a graphical user interface for displaying project information;
creating a work order, by an office user, comprising one or more tasks wherein said one or more tasks are input using the portal;
accepting said work order, by a first field user, via the portal;
performing, by said first field user, one or more tasks associated with said work order;
logging, by said first field user, completion of the one or more tasks associated with said work order via the portal;
performing, by a first lab user, one or more lab tests associated with said work order;
generating, by said first lab user, a lab report;
reviewing, by a second lab user, the lab report;
delivering the lab report to a client via the portal.

2. The method of claim 1, wherein the one or more tasks are further defined as collecting one or more specimens from a geotechnical or construction project site.

3. The method of claim 1, further comprising, upon logging in the portal that a specimen was cast during the one or more tasks, automatically creating, by the portal, a second work order requiring pickup of the specimen and assignment of the second work order to field personnel based on qualifications or placement into an unassigned queue for later assignment.

4. The method of claim 1, wherein logging completion further comprises capturing and storing geolocation data and one or more photographs in association with the work order and specimen identifiers, with offline caching on a user device and automatic synchronization to one or more servers upon reestablishment of network connectivity.

5. The method of claim 1, further comprising verifying, via the portal, a calibration status of field testing equipment prior to performing the one or more tasks, and preventing submission of testing results if calibration verification fails.

6. The method of claim 1, wherein a specimen identifier is persistently associated with a work order identifier to maintain an auditable chain of custody from field casting through lab receipt of the specimen, testing of the specimen, reporting the results of the lab test, and delivery of the results of the lab test to the client.

7. The method of claim 2, further comprising the step of delivering one or more specimens to a lab by said first field user or a second field user.

8. The method of claim 7, wherein the step of performing, by a first lab user, one or more lab tests associated with said work order is further defined as performing a concrete strength test.

9. The method of claim 7, wherein the step of performing, by a first lab user, one or more lab tests associated with said work order is further defined as performing a proctor test on a soil specimen.

10. The method of claim 7, further comprising automatically notifying clients of completion of work order tasks via the portal.

11. A system for providing multi-point connectivity among geotechnical or construction materials project stakeholders, the system comprising:
one or more servers having at least one processor;
a memory in communication with the at least one processor, said memory comprising computer executable instructions;
at least one user device in communication with said one or more servers via a data network, said at least one user device for field personnel to input data;
wherein said at least one processor is configured to execute said computer executable instructions to perform the following functions:
provide a portal having a graphical user interface for displaying project information and communication tools;
receive a plurality of data representative of a work order;
generate a notification of the work order;
receive input information indicating completion of the work order;
generate a report; and
deliver said report to a client.

12. The system of claim 11, wherein the portal comprises role-based access control with session tokens comprising JSON Web Tokens that comprise user identity and role claims, and wherein client users are only authorized to view approved final reports.

13. The system of claim 11, wherein the portal maintains a lab testing queue that automatically populates specimens for testing on a test date computed from a cast date and test age, flags overdue tests, and supports bulk data entry and completion flags.

14. The system of claim 11, wherein the at least one user device enables field personnel to collect data when the user device is not connected to said data network, and to automatically synchronize the collected data with the system upon reconnection with the data network.

15. The system of claim 14, wherein said portal further provides capabilities for:
visualizing and analyzing spatial data related to projects using a Geographic Information System (GIS) integration module;
generating customizable reports;
reviewing said generated customizable reports; and
approving said generated customizable reports; and
transmitting said approved generated customizable reports to a client.

16. The system of claim 11, wherein interfacing with third-party databases includes utilizing Application Programming Interfaces (APIs) for data exchange.

17. A non-transitory computer readable medium for providing multi-point connectivity among geotechnical or construction materials project stakeholders, the computer readable medium having computer-readable instructions stored therein that when executed by a processor performs the steps of:
receive, by the processor, a plurality of data representative of work order information;
generate, by the processor, a notification of the work order;
receive, by the processor, input information indicating completion of the work order;
receive, by the processor, report information;
deliver, by the processor, report information.

18. The non-transitory computer-readable medium of claim 17, wherein the instructions further cause the processor to transfer approved billing data associated with final report approval to generate client notifications upon report distribution via the portal.
